(19) United States
(12) Patent Application Publication
(10) Pub. No.: US 2005/0220887 A1
(43) Pub. Date: Oct. 6, 2005

(54) Method for milling frozen microparticles

(75) Inventors: Paul F. Herbert, Wayland, MA (US); Gregory C. Troiano, Weymouth, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Alkermes Controlled Therapeutics, Inc., Cambridge, MA (US)

(21) Appl. No.: 11/039,707

(22) Filed: Jan. 19, 2005

Related U.S. Application Data
(60) Provisional application No. 60/537,743, filed on Jan. 20, 2004.

(51) Int. Cl. A61K 9/14

(52) U.S. Cl. 424/489; 264/5

(57) ABSTRACT

A method for forming microparticles includes fragmenting solid particles that include a biologically active agent, a biocompatible polymer and a solvent, thereby producing fragmented solid particles, and separating the solvent from the fragmented solid particles, thereby forming the microparticles. The method can also include the steps of forming a mixture of the biologically active agent, the biocompatible polymer and the solvent, and freezing the mixture to form the solid particles. The present invention also relates to methods for producing injectable pharmaceutical compositions that include an injectable microparticle population.

### RELATED APPLICATION

This application claims the benefit of U.S. Provisional Application No. 60/537,743, filed Jan. 20, 2004. The entire teachings of the above application are incorporated herein by reference.

### BACKGROUND OF THE INVENTION

Many illnesses or conditions require administration of a constant or sustained level of an active agent to provide the desired prophylactic, therapeutic, or diagnostic effect. This can be accomplished through a multiple dosing regimen or by employing a system that releases the active agent in a sustained fashion.

Attempts to sustain medication levels include the use of biodegradable compositions, such as biocompatible polymers having incorporated therein one or more active agents. The use of these biodegradable polymer/active agent compositions, for example, in the form of microparticles or microcarriers, can provide sustained release of active agents by utilizing the inherent biodegradability of the polymer. The ability to provide a sustained level of the active agent can result in improved patient compliance and therapeutic effects.

Biodegradable polymer/active agent compositions can be produced by spraying a mixture of biocompatible polymer, solvent, and active agent into or near a cryogenic fluid such as, for example, liquid nitrogen to produce frozen microparticles. Subsequently, the microparticles can be administered to a patient as part of a pharmaceutical composition, e.g., an injectable pharmaceutical composition. However, this method of spray freezing microparticles can produce a broad range of microparticle sizes which makes administration of a pharmaceutical composition containing the microparticles difficult, if not impossible. In general, injectable pharmaceutical compositions containing the biodegradable polymer/active agent compositions, e.g., microparticles, should contain particles appropriately sized for injectability. For example, forming the pharmaceutical composition can include size-separating, e.g., sieving, microparticles produced by spray freezing. Thus, size-separation can be used to remove large particles that can cause syringe-needle blockage during injection. However, relatively large quantities of microparticles unsuitable for administration by injection can be present in the spray frozen microparticles. These particles, containing the active agent and unsuitable for injection, are typically discarded or subjected to processes for recovery of the active agent. Since the above-described spray freezing and size-separation processes can produce a low yield of microparticles suitable for injection, manufacturing costs including materials, capital equipment, utility and labor can be high.

In view of the above, improved methods and apparatus for the formation of microparticles and pharmaceutical compositions containing the microparticles are needed.

### SUMMARY OF THE INVENTION

The present invention relates to methods and apparatus for forming microparticles containing a biologically active agent for delivery to a subject in need thereof. In one embodiment, the microparticles are formulated for sustained release of the biologically active agent. The microparticles contain a biocompatible polymer having the biologically active agent incorporated therein. The biologically active agent can be a therapeutic, prophylactic and/or diagnostic agent. The invention also relates to methods for producing injectable pharmaceutical compositions that include an injectable microparticle population.

One method for forming microparticles includes the steps of fragmenting solid particles that include a biologically active agent, a biocompatible polymer, and a solvent, thereby producing fragmented solid particles; and separating the solvent from the fragmented solid particles, thereby forming the microparticles. In an additional embodiment, the method also includes the steps of first forming a mixture including the biologically active agent, the biocompatible polymer and the solvent, and then atomizing the mixture to form droplets and freezing the droplets, thereby producing the solid particles.

In another embodiment, the present invention includes a method for producing an injectable pharmaceutical composition. That method can include forming a mixture including a biologically active agent, a biocompatible polymer and a solvent. The mixture can be atomized to produce droplets and then the droplets can be frozen, thereby producing solid particles. The solid particles are fragmented, thereby forming fragmented solid particles. Then the solvent is separated from the fragmented solid particles, thereby forming microparticles. Microparticles unsuitable for administration by injection can be then size-separated from the microparticles, thereby forming an injectable microparticle population. Finally, a mixture can be formed of the injectable microparticle population and a physiologically acceptable diluent, thereby forming the injectable pharmaceutical composition.

In one aspect, the present invention also relates to an apparatus for producing microparticles. For example, the apparatus can include a solid particle production section, a fragmentation section and an extraction section, wherein the solid particle production section is joined in fluid communication with the fragmentation section and the fragmentation section is joined in fluid communication with the extraction section. The solid particle production section can include a fluid atomizer, at least one port for introducing a cryogenic fluid, and a spray chamber. The fragmentation section can include solid particle fragmentation means. The extraction section can include an extraction vessel containing a polymer non-solvent.

Practice of the present invention can produce microparticles suitable for administration to a patient, for example, by injection. The methods for producing microparticles described herein result in greater yields of administrable microparticles during size separation processes. For example, during sieving processes, greater yields of microparticles suitable for injection can be obtained. Greater yields of microparticles suitable for injection can reduce the quantity of materials, e.g., biologically active agent, needed to produce a given quantity of administrable microparticles. Thus, the present invention can reduce costs associated with the disposal of unadministrable microparticles and/or with the recovery of the active agent from unadministrable microparticles.

In addition, practice of the methods described herein for forming microparticles can maintain the morphology, density, and/or release characteristics of the resulting microparticles while increasing the yield of injectable microparticles as compared to other methods such as those that do not include fragmentation of particles or that include fragmentation of particles following separation of a solvent from the particles.

Advantageously, methods of the present invention can be performed under closed and/or sterile conditions. For example, in one embodiment, microparticles having a more desirable particle size distribution can be formed entirely within the apparatus described herein and illustrated in FIG. 1. In some embodiments, the solid particles can be fragmented within a liquid, e.g. a cryogenic fluid, in which they are formed, or the solid particles can be fragmented in the medium that is subsequently used to separate the solvent from the fragmented solid particles, e.g., a polymer non-solvent. Thus, in some embodiments, there is no need to dry or separate the solid particles from a process substance, e.g., a cryogenic fluid or a polymer non-solvent, prior to fragmentation.

Practice of the present invention allows economic manufacture of microparticles suitable for administration using smaller delivery devices, e.g., smaller diameter syringes for injection, than are currently economically feasible. By using smaller syringes to administer the microparticles to a patient, injection pain and/or adverse reaction at the injection site can be reduced.

The methods and apparatus described herein also can produce microparticles that have no significant increase in the quantities of fine particles such as particles having a particle size of less than about 20 microns. Other methods of fragmentation can produce excessive quantities of undesirable fine particles.

Methods of the present invention can use conditions, such as low temperatures, that preserve the biological activity of sensitive active agents such as temperature sensitive biologically active agents. Thus, the methods described herein are particularly suitable for producing microparticles containing thermally labile biologically active agents such as many proteins, polypeptides, and polynucleotides. Thermally labile biologically active agents include active agents that lose a substantial amount of activity when warmed to elevated temperatures, such as temperatures greater than physiological temperatures, e.g., about 37° C.

The methods and apparatus described herein provide for efficient, facile and cost effective preparation of microparticles having desirable physical and chemical properties. For example, microparticles for sustained release of a biologically active agent can be economically manufactured through practice of the present invention.

### BRIEF DESCRIPTION OF THE DRAWINGS

FIG. 1 is a view, partially in cross-section, of an apparatus suitable for continuous production of microparticles containing a biocompatible polymer and a biologically active agent incorporated therein.

FIG. 2 shows the in vivo pharmacokinetic profiles resulting from administration to Sprague-Dawley rats of milled and unmilled microparticles containing human growth hormone.

FIG. 3 is a typical ejection force profile for control microparticles directed through a 21 gauge, 1 inch long syringe.

FIG. 4 is a typical ejection force profile for microparticles produced in accordance with the present invention directed through a 21 gauge, 1 inch long syringe.

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

### DETAILED DESCRIPTION OF THE INVENTION

A description of preferred embodiments of the invention follows. The features and other details of the method of the invention will now be more particularly described with reference to the accompanying drawings and pointed out in the claims. It will be understood that the particular embodiments of the invention are shown by way of illustration and not as limitations of the invention. The principal features of this invention can be employed in various embodiments without departing from the scope of the invention.

In one embodiment, the present invention relates to a method for forming microparticles comprising the steps of: (a) fragmenting solid particles that include a biologically active agent, a biocompatible polymer, and a solvent, thereby producing fragmented solid particles; and (b) separating the solvent from the fragmented solid particles, thereby forming the microparticles. The method can also comprise the steps of forming a mixture of the biologically active agent, the biocompatible polymer, and the solvent; and freezing the mixture to form the solid particles. The biologically active agent can be a therapeutic, prophylactic and/or diagnostic agent, also referred to herein as an “active agent.”

“Microparticles,” as that term is used herein, includes a biocompatible polymer having an biologically active agent incorporated therein. The biocompatible polymer can include, for example, poly(lactic acid) or a poly(lactic acid-co-glycolic acid) copolymer. The biologically active agent can include, for example, a therapeutic, prophylactic and/or diagnostic agent such as a protein, peptide, nucleic acid or small organic molecule. The microparticles can be used to deliver the biologically active agent to a patient in need thereof, for example, in a sustained manner.

The microparticles can be of any shape, for example, spherical, non-spherical or irregular shape, and are suitable for administration by any means (e.g., by needle, needle-free delivery, or inhalation). In one embodiment, the microparticles can have a particle size from about 1 micron to about 1000 microns. The microparticles can be homogeneous or heterogeneous, for example, the microparticles can have a homogeneous or heterogeneous distribution of the biologically active agent. The microparticles can further include excipients such as, for example, surfactants, carbohydrates (e.g., monosaccharides and polysaccharides), release modifying agents, stabilizers, one or more additional biologically active agents, and any combination thereof.

As used herein, the term “particle size” refers to a number median diameter or a volume median diameter as determined by conventional particle size measuring techniques known to those skilled in the art such as, for example, laser diffraction, photon correlation spectroscopy, sedimentation field flow fractionation, disk centrifugation, electrical sensing zone method, or size classification such as sieving. The “number median diameter” reflects the distribution of particles (by number) as a function of particle diameter. The “volume median diameter” is the median diameter of the volume weighted size distribution, also referred to as D_{v,50}. The volume median diameter reflects the distribution of volume as a function of particle diameter. One example of a device that can be used to measure particle size (e.g., volume median diameter) is a Coulter LS Particle Size Analyzer (e.g., Model 130) (Beckman Coulter, Inc. Fullerton, Calif.). “Particle size” can also refer to the minimum dimension of a population of particles. For example, particles that are size classified by sieving can have a minimum dimension that is no greater than the size of the holes contained in the sieve.

As used herein, the term “solid particle” is intended to refer to the physical state of a particle's components and not necessarily to the porosity of the particle. For example, a “solid particle” includes a biologically active agent, a biocompatible polymer, and a solvent, each component in a substantially solid state, e.g., frozen. In various embodiments, the solid particle can be either porous or non-porous. The solid particles can take any of a variety of forms. For example, the solid particles can be of any shape, for example, cubical, spherical, non-spherical, irregular, or a mixture thereof. In one embodiment, the solid particles are strands. The solid particles can have a particle size, e.g., a volume median particle size, of about 25 microns to about 3 or more inches, for example, about 50 microns to about 1000 microns or about 200 microns to about 1000 microns. The solid particles contain more than a residual amount of solvent. For example, the solid particles have an average concentration of at least about 10 weight percent of solvent. In some embodiments, the solid particles have an average concentration of at least about 20, 30, 40, 50, 60, 70, 80, or at least about 90 weight percent solvent. In one particular embodiment, the solid particles have an average concentration of about 30 to about 80 weight percent solvent, e.g., about 60 to about 80 weight percent solvent.

In one embodiment, the method includes the step of fragmenting solid particles that include a biologically active agent, a biocompatible polymer, and a solvent, thereby producing fragmented solid particles. Suitable fragmentation methods include, but are not limited to, grinding, shearing, shocking, shattering, granulating, pulverizing, shredding, crushing, homogenizing, and/or milling. Suitable means for fragmenting the solid particles include, but are not limited to, mills (e.g., screening mills and impact mills such as hammer mills) and homogenizers (e.g., rotor-stator homogenizers). An example of a suitable mill for fragmenting the solid particles is the Fluid Air Granumill Jr. (Fluid Air, Inc., Aurora, Ill.). In one embodiment, the solid particles are fragmented by impacting the solid particles with a rotor and passing the impacted solid particles through a screen. For example, a Fluid Air Granumill Jr. is used to fragment the solid particles, whereby the solid particles are impacted with a rotor and the impacted solid particles are passed through a screen. An example of a suitable homogenizer for fragmenting the solid particles is the Silverson L4R Homogenizer (Silverson Machines, Inc., East Longmeadow, Mass.).

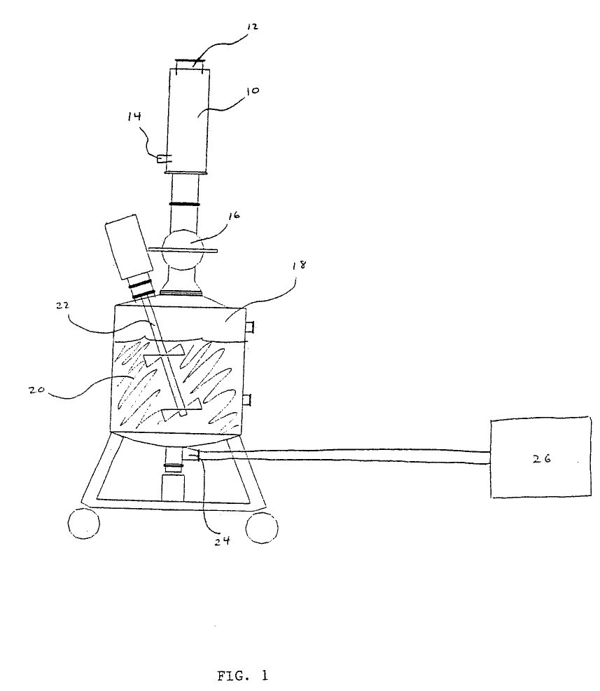

The present invention includes the use of continuous, batch, and semi-batch fragmentation processes. In a preferred embodiment, the solid particles are continuously fed to fragmentation means, e.g., a Fluid Air Granumill Jr., that is in fluid communication with a solid particle production section as illustrated in FIG. 1 and described infra.

In one embodiment, multiple-stage fragmentation can be used to fragment the solid particles. For example, solid particle fragmentation means can include two or more fragmentation devices that can be used to produce the fragmented solid particles. A particle size classifier can be used in conjunction with the fragmentation means to separate the fragmented solid particles by size. For example, a screen or sieve can be used to separate the fragmented solid particles by size prior to subsequent separation of the solvent from the fragmented solid particles.

Preferred fragmentation methods include grinding, shearing, shocking, shattering, granulating, pulverizing, shredding, crushing, homogenizing, and/or milling methods which can be performed at low temperatures. In a preferred embodiment, the solid particles are fragmented at or below the transition temperature of the solid particles. For example, fragmentation of the solid particles can be performed at a temperature below the melting point of the solvent contained in the solid particles. In some embodiments, fragmentation of the solid particles is performed at less than about 0° C., −20, −40, −60, −80, −100, −120, −140, −160, −180, or less than about −200° C. In one embodiment, the temperatures of the solid particles and the fragmented solid particles are kept below any temperature at or above which the biologically active agent would be subject to substantial degradation of its therapeutic, prophylactic, and/or diagnostic effect.

The solid particles can be dry when fragmented. Alternatively, the solid particles can be suspended in a liquid when fragmented. For example, in one embodiment, the solid particles can be suspended in a cryogenic fluid, e.g., liquid nitrogen, liquid argon, or liquid oxygen, during fragmentation. In another embodiment, the solid particles are suspended in a polymer non-solvent that is below the melting temperature of the solvent contained in the solid particles. Suitable polymer non-solvents are described infra. For example, in one embodiment, solid particles can be formed from a mixture of a biologically active agent, a biocompatible polymer, and a solvent; the solid particles can be fragmented in a polymer non-solvent, thereby forming fragmented solid particles, wherein the temperature of which is below the melting temperature of the solvent contained in the solid particles; and the temperature of the polymer non-solvent and/or the fragmented solid particles can be raised to separate the solvent from the fragmented solid particles, thereby forming the microparticles.

Preferably, the fragmented solid particles are suitable for forming microparticles, particularly injectable microparticles or an injectable microparticle population as described infra. The fragmented solid particles, for example, can have a particle size, e.g., a volume median particle size, less than or equal to about 1000 microns such as less than or equal to about 500, 400, 300, 200, 150, 125, 115, 110, 105, 100, 90, 80, 70, 60, 50, 40 or less than or equal to about 30 microns. A desired fragmented solid particle size distribution can be chosen for production of suitably sized microparticles. For example, in one embodiment, the fragmented solid particles can have a particle size less than or equal to about 106 microns. In one embodiment, the fragmented solid particles can contain about 20 or less weight percent of particles having a particle size greater than about 106 microns. For example, the fragmented solid particles can contain about 15 or less weight percent, about 10 or less weight percent, or about 5 or less weight percent of particles having a particle size greater than about 106 microns.

In one embodiment, the method also includes the step of separating the solvent from the fragmented solid particles, thereby forming the microparticles. A number of methods are known in the art and suitable for forming the microparticles by separating the solvent from the fragmented solid particles. For example, in one embodiment, fragmented solid microparticles, e.g., fragmented frozen microparticles, are contacted with a polymer non-solvent, i.e., a non-solvent of the biocompatible polymer. Thus, the solvent in the fragmented solid microparticles can be extracted as a solid and/or liquid into the polymer non-solvent, e.g., a solid or a liquid polymer non-solvent, to form microparticles that include the biocompatible polymer and the biologically active agent.

As used herein, the term “polymer non-solvent” refers to a material that essentially does not dissolve a polymer reference material, e.g., the biocompatible polymer contained in the fragmented solid particles and the microparticles.

Suitable polymer non-solvents can include, for example, ethanol, hexane, ethanol mixed with hexane, heptane, ethanol mixed with heptane, pentane, and oil. Mixing ethanol with other polymer non-solvents, such as hexane, heptane or pentane, can increase the rate of organic liquid extraction above that achieved by ethanol alone from certain biocompatible polymers such as, for example, poly(lactide-co-glycolide) polymers. Polymer non-solvent systems suitable for production of microparticles can be determined via routine experimentation using techniques well-known to those of ordinary skill in the art.

In another embodiment, some or all of the solvent contained in the fragmented solid particles is separated from the fragmented solid particles using lyophilization or vacuum drying. For example, a lyophilization or vacuum drying step can be performed prior to or following extraction of the solvent from the fragmented solid particles to remove a portion of the solvent from the fragmented solid particles or from the formed microparticles. Alternatively, lyophilization or vacuum drying can be used instead of extraction to separate the solvent from the fragmented solid particles.

Following separation of the solvent contained in the fragmented solid particles, the resulting microparticles can be filtered and dried. In one embodiment, the solvent present in the fragmented solid particles is extracted into a polymer non-solvent and the resulting microparticles are subsequently filtered from the polymer non-solvent and the microparticles are then dried. For example, a filter dryer can be used to filter and dry the microparticles. Suitable filter dryers include, but are not limited to Nutsche filter dryers. In one embodiment, the filter dryer is jacketed for temperature control, e.g., a jacketed Nutsche filter dryer can be used. The filter dryer can also be designed for vacuum drying of the microparticles. One suitable filter dryer has been custom manufactured by ITT Sherotec (Simi Valley, Calif.). Other sources for suitable filter dryers or dryer components include Martin Kurz & Co., Inc. (Mineola, N.Y.), Pope Scientific Inc. (Saukville, Wis.), and National Filter Media Corporation (Salt Lake City, Utah). Lyophilization can be used to remove substances, e.g., residual polymer non-solvent, from the microparticles.

Preferably, the microparticles contain a substantial population of injectable microparticles. The formed microparticles can be subsequently size separated to produce a fraction of microparticles unsuitable for administration by injection and a fraction of injectable microparticles, e.g., an injectable microparticle population.

“Injectable microparticles” and an “injectable microparticle population” refer to a collection of microparticles suitable for administration via injection to a patient in need of the biologically active agent contained therein. In one embodiment, the injectable microparticles can have a particle size from about 1 micron to about 1000 microns. For example, the injectable microparticles can have a particle size of less than or equal to about 1000 microns such as less than or equal to about 500, 400, 300, 200, 150, 125, 115, 110, 105, 100, 90, 80, 70, 60, 50, 40 or less than or equal to about 30 microns.

The desired injectable microparticles' particle size can be chosen for compatibility with the device used to administer the microparticles to a patient. A device used to administer the microparticles to a patient via injection can be selected based such factors as the injection type, the location of injection, the composition of the injected materials, and the volume of injection. For example, the device used to administer the microparticles can be a syringe equipped with a needle, e.g., an about 25 gauge needle to an about 19 gauge needle. In one embodiment, the injectable microparticles can be delivered with a 21 gauge needle and can have a particle size of less than or equal to about 106 microns. In one embodiment, the microparticles can contain about 20 or less weight percent of particles having a particle size greater than about 106 microns. For example, the microparticles can contain about 15 or less weight percent, about 10 or less weight percent, or about 5 or less weight percent of microparticles having a particle size greater than about 106 microns.

In one embodiment, the method of the present invention includes the step of forming a mixture of the biologically active agent, the biocompatible polymer, and the solvent. The components of the mixture may be combined in a number of ways. In one embodiment, the biocompatible polymer is mixed with the solvent prior to addition of the active agent. In another embodiment, the active agent and the solvent are mixed prior to addition of the biocompatible polymer. In another embodiment, the active agent and the biocompatible polymer are mixed prior to addition of the solvent. In yet another embodiment, the biocompatible polymer, the active agent, and the solvent are mixed together substantially concurrently.

The solvent can act to dissolve the biologically active agent at least partially or, alternatively, the solvent can dissolve essentially none of the active agent. In one embodiment, the solvent is used to dissolve, partially or completely, the biocompatible polymer in forming the mixture from which the solid particles are formed. For example, the biologically active agent can be in solution and/or suspended in the mixture.

As used herein, a “solution” is a mixture of one or more substances, referred to as the solute(s), dissolved in one or more other substances, referred to as the solvent(s).

The mixture can contain at least about 10 weight percent of solvent. In some embodiments, the mixture can contain at least about 20, 30, 40, 50, 60, 70, 80, or at least about 90 weight percent solvent. In one particular embodiment, the mixture contains about 30 to about 80 weight percent solvent, e.g, about 60 to about 80 weight percent solvent.

The method for producing microparticles can also include the step of forming solid particles from the mixture of the biologically active agent, the biocompatible polymer, and the solvent. Solid particles can be formed by freezing the mixture containing the biocompatible polymer, the biologically active agent, and the solvent. In one embodiment, the mixture is frozen by bulk freezing. For example, the mixture may be frozen to form solid particles that include large pieces such as pieces having a particle size of about 500 microns to about 3 or more inches. In one embodiment, the solid particles predominantly include large pieces of the frozen mixture. In another embodiment, the mixture can be frozen by, for example, pouring, dripping, atomizing, or extruding the mixture into or near a liquid or vapor polymer non-solvent which is at a temperature below the freezing point of the mixture or a cryogenic fluid such as liquid nitrogen or liquid argon. The mixture can be frozen into solid particles, for example, from droplets or as strands of the frozen mixture.

In one embodiment, freezing the mixture to form the solid particles includes processing the mixture to form droplets, e.g., microdroplets, and freezing the droplets. In a preferred embodiment, a significant portion of the droplets contains the biocompatible polymer, biologically active agent and the solvent. The droplets can be formed using any of a variety of means known in the art. Examples of means for forming the droplets include atomizing the mixture such as by directing the mixture through a nozzle or jet such as a pressure nozzle, an ultrasonic nozzle, or a Rayleigh jet or by other known means for creating droplets from a mixture. In one embodiment, means for processing the mixture to form droplets includes a two-fluid nozzle. In some embodiments using two-fluid nozzles, the two-fluid nozzle includes an air cap containing one or more orifices, in addition to one or more orifices through which droplets are formed, to provide for flow of gas from the nozzle. The presence of one or more additional orifices in the air cap can increase the flow of gas through the nozzle.

The droplets can be frozen by exposing the droplets to a liquid or gas, e.g., a polymer non-solvent, which is at a temperature below the freezing point of the mixture or by exposing the droplets to a cryogenic fluid such as liquid nitrogen, liquid argon, or liquid oxygen.

A wide range of sizes of solid particles can be made by varying the droplet size, for example, by changing the nozzle diameter or by varying the viscosity of the mixture. In one embodiment, the solid particles can have a particle size of less than or equal to about 200 microns prior to fragmenting. For example, the particle size of the solid particles can be about 100 to about 200 microns.

In one embodiment, the mixture is frozen into solid particles as strands. For example, the method can include the additional steps of forming a mixture of the biologically active agent, the biocompatible polymer and the solvent, and freezing the mixture to form the solid particles wherein freezing the mixture to form the solid particles includes forming frozen strands of the mixture, thereby forming the solid particles. Freezing the mixture into solid particle strands can be accomplished using any of a number of techniques known in the art. For example, the mixture can be forced through an orifice into strands and subsequently or concurrently frozen. In one embodiment, high molecular weight biocompatible polymer is present in the mixture used to produce the strands.

Descriptions of suitable biologically active agents, biocompatible polymers, and solvents of the solid particles and of the mixtures from which the solid particles can be formed follow.

The term “biologically active agent,” as used herein, is an agent or its pharmaceutically acceptable salt which, when released in vivo, possesses the desired biological activity, for example, therapeutic, diagnostic and/or prophylactic properties. The term “biologically active agent” includes stabilized biologically active agents such as described infra. The terms “biologically active agent” and “active agent” are used interchangeably herein.

Examples of suitable biologically active agents include, but are not limited to, proteins, muteins and active fragments thereof, such as immunoglobulins, antibodies, cytokines (e.g., lymphokines, monokines, chemokines), interleukins, interferons (β-IFN, α-IFN and γ-IFN), erythropoietin, nucleases, tumor necrosis factor, colony stimulating factors, insulin, enzymes (e.g., superoxide dismutase, tissue plasminogen activator), tumor suppressors, blood proteins, hormones and hormone analogs (e.g., growth hormone (e.g., human growth hormone), follicle stimulating hormone, adrenocorticotropic hormone, luteinizing hormone releasing hormone (LHRH), GLP-1 and exendin), vaccines (e.g., tumoral, bacterial and viral antigens), antigens, blood coagulation factors; growth factors; peptides such as protein inhibitors, protein antagonists, and protein agonists; nucleic acids, such as antisense molecules; oligonucleotides; ribozymes and derivatives (e.g., pegylated derivatives) thereof. Both naturally occurring and synthetic biologically active agents are suitable for use in the present invention.

Additional biologically active agents suitable for use in the invention include, but are not limited to, antipsychotic agents such as aripiprazole, risperidone, and olanzapine; antitumor agents such as bleomycin hydrochloride, carboplatin, methotrexate and adriamycin; antibiotics such as gentamicin, tetracycline hydrochloride and ampicillin; antipyretic, analgesic and anti-inflammatory agents; antitussives and expectorants such as ephedrine hydrochloride, methylephedrine hydrochloride, noscapine hydrochloride and codeine phosphate; sedatives such as chlorpromazine hydrochloride, prochlorperazine hydrochloride and atropine sulfate; muscle relaxants such as tubocurarine chloride; antiepileptics such as sodium phenyloin and ethosuximide; antiulcer agents such as metoclopramide; antidepressants such as clomipramine; antiallergic agents such as diphenhydramine; cardiotonics such as theophillol; antiarrhythmic agents such as propranolol hydrochloride; vasodilators such as diltiazem hydrochloride and bamethan sulfate; hypotensive diuretics such as pentolinium and ecarazine hydrochloride; antidiuretic agents such as metformin; anticoagulants such as sodium citrate and sodium heparin; hemostatic agents such as thrombin, menadione sodium bisulfite and acetomenaphthone; antituberculous agents such as isoniazide and ethanbutol; hormones such as prednisolone sodium phosphate and methimazole; and narcotic antagonists such as nalorphine hydrochloride.

In one embodiment, the biologically active agent is at least one member selected from the group consisting of proteins, immunoglobulin proteins, interleukins, interferons, erythropoietin, antibodies, cytokines, hormones, antigens, growth factors, nucleases, tumor enzymes, tumor suppression genes, antisense molecules, antibiotics, anesthetics, sedatives, cardiovascular agents, antitumor agents, antineoplastics, antihistamines and vitamins.

In one embodiment, the biologically active agent is stabilized. The biologically active agent can be stabilized against degradation, loss of potency and/or loss of biological activity, all of which can occur during formation of the microparticles having the biologically active agent dispersed therein, and/or prior to and during in vivo release of the biologically active agent from the microparticles. In one embodiment, stabilization can result in a decrease in the solubility of the biologically active agent, the consequence of which is a reduction in the initial release of the biologically active agent, in particular, when release is from microparticles for sustained release of the biologically active agent. In addition, the period of release of the biologically active agent from the microparticles can be prolonged.

Stabilization of the biologically active agent can be accomplished, for example, by the use of a stabilizing agent or a specific combination of stabilizing agents. “Stabilizing agent,” as that term is used herein, is any agent which binds or interacts in a covalent or non-covalent manner or is included with the biologically active agent. Stabilizing agents suitable for use in the invention are described in U.S. Pat. Nos. 5,716,644 and 5,674,534 to Zale, et al.; U.S. Pat. Nos. 5,654,010 and 5,667,808 to Johnson, et al.; U.S. Pat. No. 5,711,968 to Tracy, et al., and U.S. Pat. No. 6,265,389 to Burke, et al.; and U.S. Pat. No. 6,514,533 to Burke, et al., the entire teachings of each of which are incorporated herein by reference.

For example, a metal cation can be complexed with the biologically active agent, or the biologically active agent can be complexed with a polycationic complexing agent such as protamine, albumin, spermidine and spermine, or associated with a “salting-out” salt. In addition, a specific combination of stabilizing agents and/or excipients may be needed to optimize stabilization of the biologically active agent. For example, when the biologically active agent is an acid-stable or free sulfhydryl-containing protein such as β-IFN, a particular combination of stabilizing agents which includes a disaccharide and an acidic excipient can be added to a mixture prior to formation of the microparticles. This type of stabilizing formulation is described in detail in U.S. Pat. No. 6,465,425 issued to Tracy, et al., on Oct. 15, 2002, the entire contents of which is incorporated herein by reference.

Suitable metal cations include any metal cation capable of complexing with the biologically active agent. A metal cation-stabilized biologically active agent, as described herein, includes a biologically active agent and at least one type of metal cation wherein the cation is not significantly oxidizing to the active agent. In a particular embodiment, the metal cation is multivalent, for example, having a valency of +2 or more. If the agent is metal cation-stabilized, it is preferred that the metal cation is complexed to the biologically active agent.

Suitable stabilizing metal cations include biocompatible metal cations. A metal cation is biocompatible if the cation is non-toxic to the patient in a therapeutic, prophylactic or diagnostic dosage and also presents essentially no deleterious or untoward effects on the patient's body, such as a significant immunological reaction at the injection site. The suitability of metal cations for stabilizing biologically active agents and the ratio of metal cation to active agent needed can be determined by one of ordinary skill in the art by performing a variety of stability-indicating techniques such as polyacrylamide gel electrophoresis, isoelectric focusing, reverse phase chromatography, and High Performance Liquid Chromatography (HPLC) analysis on particles of metal cation-stabilized biologically active agents, for example, prior to and following microparticle formation, fragmentation of the microparticles, and/or size-separation of the microparticles. The molar ratio of metal cation to biologically active agent is typically between about 1:2 and about 100:1, preferably between about 2:1 and about 50:1.

Examples of stabilizing metal cations include, but are not limited to, K⁺, Zn⁺², Mg⁺² and Ca⁺². Stabilizing metal cations also include cations of transition metals such as Cu⁺². Combinations of metal cations can also be employed. For example, in one embodiment, Zn⁺² is used as a stabilizing metal cation for growth hormone (e.g., human growth hormone (hGH)) at a zinc cation component to hGH molar ratio of about 4:1 to about 100:1. In one embodiment, the zinc cation component to hGH molar ratio is about 4:1 to about 12:1, and most preferably 10:1. In another embodiment, Zn⁺2 is used as a stabilizing metal cation for bovine serum albumin (herein “BSA”) at a zinc cation component to BSA molar ratio of about 25:1 to about 100:1. In one embodiment, the zinc cation component to BSA molar ratio is about 50:1.

The biologically active agent can also be stabilized with at least one polycationic complexing agent. Suitable polycationic complexing agents include, but are not limited to, protamine, spermine, spermidine and albumin. The suitability of polycationic complexing agents for stabilizing active agents can be determined by one of ordinary skill in the art in the manner described above for stabilization with a metal cation. An equal weight ratio of polycationic complexing agent to biologically active agent can be suitable.

Further excipients can be added to the solid particles and microparticles of the present invention, for example, to maintain the potency of the active agent over the duration of release or to modify polymer degradation and biologically active agent release. One or more excipients can be added to the mixture which is then used to form the solid particles. For example, an excipient may be suspended or dissolved along with the biocompatible polymer and biologically active agent prior to formation of the solid particles. In addition, one or more excipients can be mixed with the microparticles, with the injectable microparticle population, or with the injectable pharmaceutical composition. For example, an excipient can be blended with the microparticles prior to the size-separation of microparticles unsuitable for administration by injection. Thus, excipient particles unsuitable for administration by injection can also be removed from the mixture of microparticles and excipient. In another embodiment, an excipient, suitably sized for administration by injection, is blended with the injectable microparticle population prior to formation of the injectable pharmaceutical composition or is blended with the injectable pharmaceutical composition.

Suitable excipients include, for example, carbohydrates, amino acids, fatty acids, surfactants, and bulking agents. Such excipients are known to those of ordinary skill in the art. An acidic or a basic excipient is also suitable. The amount of excipient used is based on its ratio to the biologically active agent, on a weight basis. For amino acids, fatty acids and carbohydrates, such as sucrose, trehalose, lactose, mannitol, dextran and heparin, the ratio of carbohydrate to biologically active agent, can be between about 1:10 and about 20:1. For surfactants, the ratio of surfactant to biologically active agent can be between about 1:1000 and about 2:1. Bulking agents typically include inert materials. Suitable bulking agents are known to those of ordinary skill in the art.

The excipient can include a metal cation component which is separately dispersed within the microparticles. This metal cation component can act to modulate the release of the biologically active agent and is not complexed with the active agent. The metal cation component can optionally contain the same species of metal cation, as is contained in the metal cation stabilized biologically active agent, if present, and/or can contain one or more different species of metal cation. The metal cation component acts to modulate the release of the biologically active agent from the microparticles and can enhance the stability of the active agent in the microparticles. A metal cation component used in modulating release typically includes at least one type of multivalent metal cation. Examples of metal cation components suitable to modulate release of the biologically active agent include or contain, for example, Mg(OH)₂, MgCO₃ (such as 4MgCO₃.Mg(OH)₂.5H₂O), MgSO₄, Zn(OAc)₂, Mg(OAc)₂, ZnCO₃ (such as 3Zn(OH)₂.2ZnCO₃)ZnSO₄, ZnCl₂, MgCl₂, CaCO₃, Zn₃(C₆H₅O₇)₂ and Mg₃(C₆H₅O₇)₂. A suitable ratio of metal cation component to biocompatible polymer includes between about 1:500 to about 1:2 by weight. The optimum ratio can depend upon the biocompatible polymer and the metal cation component utilized and can be determined by one of ordinary skill in the art without undue experimentation. A polymer composition containing a dispersed metal cation component to modulate the release of an active agent from the polymer composition is further described in U.S. Pat. No. 5,656,297 issued to Bernstein, et al., on Aug. 12, 1997, and U.S. Pat. No. 5,912,015 issued to Bernstein, et al., on Jun. 15, 1999, the entire contents of both of which are incorporated herein by reference.

In yet another embodiment, at least one pore forming agent, such as a water soluble salt, sugar or amino acid, is included in the mixture of the biologically active agent, the biocompatible polymer, and the solvent to modify the microstructure of the subsequently produced microparticles. The proportion of pore forming agent added to the mixture can be, for example, about 0.1% (w/w) to about 30% (w/w).

The microparticles prepared according to the present invention can contain from about 0.01% (w/w) to about 90% (w/w) of the biologically active agent (based on dry weight of the microparticles). The amount of biologically active agent can vary depending upon the desired effect of the agent, the planned release levels, and the time span over which the agent is to be released. A preferred range of biologically active agent loading is about 0.1% (w/w) to about 75% (w/w), for example, about 0.1% (w/w) to about 60% (w/w), about 0.5% (w/w) to about 40% (w/w), about 0.5% (w/w) to about 20% (w/w) or about 0.5% (w/w) to about 15% (w/w).

Polymers used in the formulation of the microparticles described herein include any polymer which is biocompatible. Biocompatible polymers suitable for use in the present invention include biodegradable and non-biodegradable polymers and blends and copolymers thereof, as described herein. A polymer is biocompatible if the polymer and any degradation products of the polymer are non-toxic to the patient and also possess no significant deleterious or untoward effects on the patient's body, such as a significant immunological reaction at an injection or implantation site.

“Biodegradable,” as defined herein, means the composition will degrade or erode in vivo to form smaller chemical species. Degradation can result, for example, by enzymatic, chemical and physical processes. Suitable biocompatible, biodegradable polymers include, for example, poly(lactides), poly(glycolides), poly(lactide-co-glycolides), poly(lactic acid)s, poly(glycolic acid)s, polycarbonates, polyesteramides, polyanydrides, poly(amino acids), polyorthoesters, poly(dioxanone)s, poly(alkylene alkylate)s, copolymers or polyethylene glycol and polyorthoester, biodegradable polyurethane, blends thereof, and copolymers thereof.

Suitable biocompatible, non-biodegradable polymers include non-biodegradable polymers such as, for example, polyacrylates, polymers of ethylene-vinyl acetates and other acyl substituted cellulose acetates, non-degradable polyurethanes, polystyrenes, polyvinylchloride, polyvinyl flouride, poly(vinyl imidazole), chlorosulphonate polyolefins, polyethylene oxide, blends thereof, and copolymers thereof, such as PLG-co-EMPO described in U.S. patent application Ser. No. 09/886,394 entitled “Functionalized Degradable Polymer” and filed on Jun. 22, 2001, the entire contents of which is hereby incorporated by reference.

Further, the terminal functionalities or pendant groups of the biocompatible polymers can be modified, for example, to modify hydrophobicity, hydrophilicity and/or to provide, remove or block moieties which can interact with the biologically active agent via, for example, ionic or hydrogen bonding.

In one embodiment, the biocompatible polymer is at least one member selected from the group consisting of poly(lactide)s, poly(glycolide)s, poly(lactide-co-glycolide)s, poly(lactic acid)s, poly(glycolic acid)s, polycarbonates, polyesteramides, polyanhydrides, poly(amino acids), polyorthoesters, polyacetals, polycyanoacrylates, polyetheresters, polycaprolactone, poly(dioxanone)s, poly(alkylene alkylate)s, polyurethanes, and blends and copolymers thereof.

In a preferred embodiment of the present invention, the polymer used is a poly(lactic acid-co-glycolic acid) (“PLG”) copolymer. The poly(lactic acid-co-glycolic acid)polymer includes d-, l-, or racemic forms of the polymer, for example, in some embodiments the polymer used is poly(d,l-lactic acid-co-glycolic acid). In some embodiments, the poly(lactic acid-co-glycolic acid) contains free carboxyl end groups. In other embodiments, the poly(lactic acid-co-glycolic acid) contains alkyl ester end groups such as methyl ester end groups.

Acceptable molecular weights for biocompatible polymers used in this invention can be determined by a person of ordinary skill in the art taking into consideration factors such as the desired polymer degradation rate, physical properties such as mechanical strength, and the rate of dissolution of polymer in the solvent. Typically, an acceptable range of molecular weight is of about 2,000 Daltons to about 2,000,000 Daltons. In a preferred embodiment, the polymer is a biodegradable polymer or copolymer. In another preferred embodiment, the polymer is a poly(lactide-co-glycolide) which can have lactide:glycolide ratios of about 25:75 to about 85:15 such as about 25:75, 50:50, 75:25 and 85:15, and a molecular weight of about 5,000 Daltons to about 150,000 Daltons. In one embodiment, the molecular weight of the PLG has a molecular weight of about 5,000 Daltons to about 42,000 Daltons.

Suitable solvents, e.g., polymer solvents, suitable for production of microparticles can be determined via routine experimentation using techniques well-known to those of ordinary skill in the art. Suitable solvents include, but are not limited to, methylene chloride, acetone, acetic acid, ethyl acetate, methyl acetate, tetrahydrofuran, dimethylsulfoxide (DMSO), methyl ethyl ketone (MEK), acetonitrile, toluene, and chloroform. In one embodiment, the solvent is selected from the group consisting of methylene chloride, chloroform, ethyl acetate, methyl acetate, acetone, acetic acid, acetonitrile, dimethylsulfoxide, methyl ethyl ketone and toluene.

The concentration of the biologically active agent in the mixture can be, for example, about 0.01 to about 100 g/L. The exact quantity of active agent can be determined based on the desired dosage of the biologically active agent from the microparticles, the desired period of active agent release, and the medical or veterinary condition being treated or diagnosed. For example, in one embodiment, the active agent is hGH and the concentration of biologically active agent in the mixture from which the solid particles are formed can be from about 10 to about 50 μL, e.g., about 20 to about 40 g/L.

Methods for forming a microparticles containing a biologically active agent and suitable components thereof are described in U.S. Pat. No. 5,019,400, issued to Gombotz, et al., on May 28, 1991; U.S. Pat. No. 5,922,253 issued to Herbert, et al., on Jul. 13, 1999; and U.S. Pat. No. 6,455,074 issued to Tracy, et al., on Sep. 24, 2002, the entire contents of each of which are incorporated herein by reference.

The present invention further relates to the microparticles formed according to the methods described herein. The microparticles include a biocompatible polymer such as, for example, poly(lactic acid) or a poly(lactic acid-co-glycolic acid) copolymer, and a biologically active agent, for example, a therapeutic, prophylactic and/or diagnostic agent such as a protein, peptide, nucleic acid or small organic molecule. In one embodiment, the microparticles further include one or more excipients and/or release modifiers.

In an additional embodiment, the present invention includes a method for producing an injectable pharmaceutical composition comprising the steps of: (a) forming a mixture including a biologically active agent, a biocompatible polymer, and a solvent; (b) atomizing the mixture to produce droplets and freezing the droplets, thereby producing solid particles; (c) fragmenting the solid particles, thereby forming fragmented solid particles; (d) separating the solvent from the fragmented solid particles, thereby forming microparticles; (e) size-separating microparticles unsuitable for administration by injection from the microparticles, thereby producing an injectable microparticle population; and (f) forming a mixture of the injectable microparticle population and a physiologically acceptable diluent, thereby forming the injectable pharmaceutical composition.

Methods for forming a mixture including a biologically active agent, a biocompatible polymer, and a solvent; atomizing the mixture to produce droplets and freezing the droplets; fragmenting the solid particles; and separating the solvent from the fragmented solid particles are described supra.

In one embodiment, the microparticles unsuitable for administration by injection are size-separated from the microparticles, thereby producing an injectable microparticle population. For example, the microparticles having a particle size suitable for administration to a patient, e.g., by injection, can be separated from those particles unsuitable for administration by injection, e.g., those particles that are too large for practical injection. In one embodiment, a screen or sieve can be used to size-separate the microparticles unsuitable for administration by injection from the microparticles. In one embodiment, the injectable microparticle population can have a particle size of less than or equal to about 106 microns. In one embodiment, the microparticles unsuitable for administration by injection can represent about 20 or less percent of the total weight of the microparticles. For example, the microparticles unsuitable for administration by injection represent can represent about 15 or less percent, about 10 or less percent, or about 5 or less percent of the total weight of the microparticles.

In one embodiment, the method includes forming a mixture of the injectable microparticle population and a physiologically acceptable diluent, thereby forming the injectable pharmaceutical composition. The injectable microparticles can be mixed with one or more physiologically acceptable diluents using techniques well-known in the art. In addition to the physiologically acceptable diluent, the pharmaceutical compositions described herein may also include other pharmaceutically acceptable excipients such as, for example, stabilizers and delivery vehicles. Pharmaceutically acceptable excipients can be selected by one of ordinary skill in the art without undue experimentation. Compositions for the administration of microparticles are described, for example, in U.S. Pat. No. 6,495,164 issued to Ramstack, et al., on Dec. 17, 2002. One example of a suitable physiologically acceptable diluent is 3% carboxymethylcellulose (low viscosity) and 0.1% TWEEN® 20 in 0.9% aqueous sodium chloride. Other suitable physiologically acceptable diluents include saline, sorbitol solutions and oil formulations.

In one embodiment, the microparticles of the present invention can be incorporated into an alternative pharmaceutical composition for the administration of the biologically active agent. For example, the microparticles can be formed into an implantable pharmaceutical composition such as a mass of the microparticles. In one embodiment, microparticles can be mechanically compressed to form the implantable mass of microparticles.

The present invention also relates to an apparatus for producing microparticles. In one embodiment, the invention includes an apparatus for producing microparticles, comprising: (a) a solid particle production section including a fluid atomizer, at least one port for introducing a cryogenic fluid, and a spray chamber; (b) a fragmentation section including solid particle fragmentation means; and (c) an extraction section including an extraction vessel containing a polymer non-solvent; wherein the solid particle production section is joined in fluid communication with the fragmentation section and the fragmentation section is joined in fluid communication with the extraction section.

The apparatus depicted in FIG. 1 is an example of an apparatus suitable for producing microparticles according to the methods described herein.

The solid particle production section of the apparatus as shown in FIG. 1 includes spray chamber 10, spray head 12 and optional port 14. Spray chamber 10 can be jacketed for controlling the temperature of the chamber. A feed mixture of the biocompatible polymer, the biologically active agent, and the solvent can be fed to spray chamber 10 via spray head 12. Spray head 12 can contain a fluid atomizer for atomizing the feed mixture to produce droplets, e.g., microdroplets, which fall through spray chamber 10. The mixture can be atomized by directing the mixture through a nozzle or jet such as a pressure nozzle, an ultrasonic nozzle, or a Rayleigh jet or by other known means for creating droplets from a mixture. In one embodiment, the mixture can be atomized using a two-fluid nozzle. In some embodiments using a two-fluid nozzle, the two-fluid nozzle includes an air cap containing one or more orifices, in addition to one or more orifices through which droplets are formed, to provide for flow of gas from the nozzle. The presence of one or more additional orifices in the air cap can increase the flow of gas through the nozzle.

Cryogenic fluid, e.g., liquid nitrogen, liquid argon, or liquid oxygen, can be introduced through spray head 12. Spray head 12 can at least one port for introducing cryogenic fluid to spray chamber 10. In one embodiment, spray head 12 contains at least one cryogenic fluid nozzle for introducing the fluid to spray chamber 10. Exposure of the droplets to the cryogenic fluid can cause the droplets to freeze, thereby producing solid particles. Typically, the solid particles are entrained or become entrained in a stream of the cryogenic fluid. Optional port 14 is an additional port through which cryogenic fluid can be introduced. It has been discovered that by introducing additional cryogenic fluid prior to fragmentation, e.g., via spray head 12 and/or optional port 14, particle hold-up in the fragmentation section can be reduced.

In one embodiment, spray chamber 10 has an upper-portion that includes the fluid atomizer and at least one port for introducing a cryogenic fluid, e.g., as contained in spray head 12, and a lower-portion that includes a solid particle outlet and at least one port, e.g., optional port 14, for introducing a cryogenic fluid. In one embodiment, the solid particle outlet of the spray chamber is in fluid communication with the fragmentation section.

The solid particle production section of the apparatus is joined in fluid communication with the fragmentation section. The fragmentation section of the apparatus as shown in FIG. 1 includes solid particle fragmentation means, e.g., mill 16.

Frozen solid particles produced in the solid particle production section of the apparatus are then directed into the fragmentation section. The frozen solid particles can be entrained within the cryogenic fluid. Mill 16 is in fluid connection with spray chamber 10 such that frozen solid particles entrained within the cryogenic fluid can flow into it. Mill 1 can include solid particle fragmentation means as described supra. An example of suitable fragmentation means is a modified Fluid Air Granumill Jr. Within mill 16 such as a modified Fluid Air Granumill Jr., the solid particles can be impacted with a rotor and the impacted solid particles can be passed through a screen. The Granumill Jr. can be modified to allow cleaning in place (‘CIP’) and/or sanitization via steaming in place (‘SIP’). For example, a Rulon seal can be added on the Granumill Jr. shaft that exits the motor and the motor purge stream can be modified so that it can be steamed. In addition, polytetrafluoroethylene o-rings can be added to isolate the threaded connection for attaching the rotor of the Granumill Jr. from the sterile envelope.

The frozen solid particles are milled in mill 16, thereby producing the fragmented solid particles. The fragmented solid particles are typically entrained in the cryogenic fluid. The fragmented solid particles produced in the fragmentation section of the apparatus are then directed into the extraction section.

The fragmentation section of the apparatus is joined in fluid communication with the extraction section. The extraction section of the apparatus as shown in FIG. 1 includes extraction vessel 18, polymer non-solvent 20, optional mixer 22, and outlet port 24.

Mill 16 is in fluid connection with extraction vessel 18 such that fragmented solid particles entrained within the cryogenic fluid can flow into it. Extraction vessel 18 can contain polymer non-solvent 20 as described supra. In one embodiment, the polymer non-solvent is cold ethanol, e.g., ethanol at about −112° C. to about −80° C. such as about −112° C. to about −104° C., for example, about −104° C. The fragmented solid particles are contacted with polymer non-solvent 20 to separate the solvent contained in the fragmented solid particles from the particles, e.g., the solvent is extracted from the fragmented solid particles, thereby forming the microparticles. Polymer non-solvent and the fragmented microparticles are optionally stirred using mixer 22. In one embodiment, non-solvent 20, containing the fragmented solid particles is slowly warmed to about −40° C., for example, over a time period of about 2-3 hours. For example, the solvent can be extracted from the fragmented solid particles as described in U.S. Pat. No. 6,358,443 issued to Herbert, et al., on Mar. 19, 2002, the entire contents of which are incorporated herein by reference. The cryogenic fluid can leave the system as it is transformed to a gas by contact with non-solvent 20. The microparticles, entrained in the polymer non-solvent, exit extraction vessel 18 via outlet port 24.

In one embodiment, the microparticles, entrained in the polymer non-solvent, are directed to filter/dryer 26. Filter/dryer 26 separates the microparticles from the polymer non-solvent and removes residual substances from the microparticles. In one embodiment, filter/dryer 26 is jacketed for temperature control. Filter/dryer 26 can also be designed for vacuum drying of the microparticles. For example, filter/dryer 26 can include a filter dryer. Suitable filter dryers include, but are not limited to Nutsche filter dryers. Sources for suitable filter dryers include Martin Kurz & Co., Inc. (Mineola, N.Y.), Pope Scientific Inc. (Saukville, Wis.), and National Filter Media Corporation (Salt Lake City, Utah). The resulting particles can then be size-separated as described supra.

The present invention also relates to use of the microparticles prepared according to the described methods for the manufacture of a medicament for use in therapy. The invention includes microparticles, produced according to the methods described herein, and pharmaceutical compositions that include the microparticles. Pharmaceutical compositions including the microparticles are suitable for administration to a patient.

The microparticles and microparticle-containing pharmaceutical compositions described herein can be administered in vivo, for example, to a human or to an animal, orally or parenterally such as by injection, implantation (e.g., subcutaneously, intramuscularly, intraperitoneally, intracranially, and intradermally), administration to mucosal membranes (e.g., intranasally, intravaginally, intrapulmonary, buccally or by means of a suppository), or by in situ delivery (e.g., by enema or aerosol spray) to provide the desired dosage of the biologically active agent based on the known parameters for treatment with the particular active agent of various medical conditions.

The microparticles and microparticle-containing pharmaceutical compositions of the present invention can provide sustained release of the biologically active agent contained therein. Thus, the microparticles described herein can be used to provide a therapeutically, prophylactically, and/or diagnostically effective amount of the biologically active agent to a patient for a sustained period. The microparticles formed by the method of the present invention can provide increased therapeutic, prophylactic, and/or diagnostic benefits by reducing fluctuations of the active agent concentration in blood, by providing a more desirable release profile, and by potentially lowering the total amount of biologically active agent needed to provide a therapeutic, prophylactic, and/or diagnostic benefit without the need for additional components.

As used herein, a “therapeutically effective amount,” a “prophylactically effective amount” or a “diagnostically effective amount” is the amount of the biologically active agent or the amount of microparticles containing the biologically active agent needed to elicit the desired biological, prophylactic or diagnostic response following administration of the microparticles or a microparticle-containing pharmaceutical composition.

“Sustained release,” as that term is used herein, is a release of the biologically active agent from the microparticles which occurs over a period which is longer than the period during which a biologically significant amount of the active agent would be available following direct administration of the active agent, e.g., a solution or suspension of the active agent. In one embodiment, a sustained release is a release of the biologically active agent which occurs over a period of at least about one day such as, for example, at least about 2, 4, 6, 8, 10, 15, 20, 30, 60, or at least about 90 days. A sustained release of the active agent can be a continuous or a discontinuous release, with relatively constant or varying rates of release. The continuity of release and level of release can be affected by the type of polymer composition used (e.g., monomer ratios, molecular weight, block composition, and varying combinations of polymers), biologically active agent loading, and/or selection of excipients to produce the desired effect.

“Sustained release” is also referred to in the art as “modified release,” “prolonged release,” “long acting release (‘LAR’),” or “extended release.” “Sustained release,” as used herein, also encompasses “sustained action” or “sustained effect.” “Sustained action” and “sustained effect,” as those terms are used herein, refer to an increase in the time period over which the biologically active agent performs its therapeutic, prophylactic and/or diagnostic activity as compared to an appropriate control. “Sustained action” is also known to those experienced in the art as “prolonged action” or “extended action.”

The microparticles and pharmaceutical compositions described herein can be administered using any dosing schedule which achieves the desired therapeutic, prophylactic and/or diagnostic levels for the desired period of time. For example, a sustained release pharmaceutical composition can be administered and the patient monitored until levels of the biologically active agent being delivered return to baseline. Following a return to baseline, the sustained release pharmaceutical composition can be administered again. Alternatively, the subsequent administration of the sustained release pharmaceutical composition can occur prior to achieving baseline levels in the patient.

### EXEMPLIFICATION

The invention will now be further and specifically described by the following examples which are not intended to be limiting.

### Example 1A

This example describes the production of placebo frozen, solid particles and control microparticles.

100 grams of a poly(d,l-lactide-co-glycolide)polymer having 50 mol % d,l-lactide, 50 mol % glycolide, and an acid end group (MEDISORB® 5050 DL PLG 2A polymer; Alkermes, Inc., Cincinnati, Ohio) was dissolved using 500 milliliters (mL) of methylene chloride.

The resulting mixture was then spray frozen to produce frozen, solid particles. The mixture was atomized at about 120 mL/minute through a 2-fluid nozzle with a 35 psi nitrogen gas stream (about 160 standard liters per minute) into a liquid nitrogen stream (from 4 nozzles at 30 psi). The nozzles used were as follows: 2-fluid nozzle: fluid cap 2050, air cap 70 m (modified for microparticle production by drilling 8 holes through the air cap to provide for flow of nitrogen gas through the air cap) (Spraying Systems Co., Wheaton, Ill.); and liquid nitrogen nozzles: Model No. 110015 (Spraying Systems Co., Wheaton, Ill.). The resulting frozen, solid particles were collected into a bucket of liquid nitrogen.

A portion of the frozen, solid particles was placed into a container of frozen ethanol wherein the ratio of ethanol to methylene chloride was about 10:1 to about 20:1. The container was stored in a freezer at −80° C. for at least overnight, e.g., usually for about 1 day but for as much as 4 days, after which the resulting microparticles were filtered from the ethanol. The microparticles were then placed overnight in a lyophilizer (Model No. ElNB352EBCB, Kinetics FTS Systems, Stone Ridge, N.Y.). The resulting lyophilized microparticles served as an unmilled control sample.

Other portions of the frozen, solid particles were collected and suspended in liquid nitrogen at a concentration of about 100 grams of frozen, solid particles per 5 liters of liquid nitrogen. Various batches of these suspended frozen, solid particles were produced and milled or homogenized as described in Examples 1B and 1C, infra.

### Example 1B

This example describes the homogenization of placebo frozen, solid particles.

About 12.5 grams of frozen, solid particles suspended in about 1 liter of liquid nitrogen, prepared as described in Example 1A, in a 1 liter beaker were homogenized using a Silverson L4R Homogenizer (Silverson Machines, Inc.; East Longmeadow, Mass.) at about 10,000 rpm for about 30 seconds. The resulting homogenized frozen, solid particles were filtered from the liquid nitrogen, placed in frozen ethanol, filtered from the ethanol, and lyophilized as described in Example 1A to produce homogenized microparticles.

The particle size distributions of the homogenized and unmilled control microparticles were then determined using a Coulter LS Particle Size Analyzer (Model 130, Beckman Coulter, Inc. Fullerton, Calif.).

Two batches each of unmilled control and homogenized microparticles were produced. The unmilled control microparticle batches had volume median particle sizes of 77.0 microns, with 21.4 weight percent above 106 microns, and 51.0 microns, with 16.7 weight percent above 106 microns. The homogenized microparticle batches had volume median particle sizes of 53.0 microns, with 17.8 weight percent above 106 microns, and 33.7 microns, with 14.0 weight percent above 106 microns, respectively. Thus, an average of about 24.5 weight percent of the unmilled control microparticles were larger than 106 microns, while an average of about 15.9 weight percent of the homogenized microparticles were larger than 106 microns. Of the two trials using the Silverson homogenizer, both showed a reduction in the percentage of microparticles that were larger than 106 microns.

### Example 1C

The following example describes the milling of placebo frozen, solid particles.

About 250 grams of frozen, solid particles suspended in about 5 liters of liquid nitrogen, prepared as described in Example 1A, were milled using a Granumill Jr. (Fluid Air, Inc.; Aurora, Ill.) equipped with a screen (Fluid Air Part No. 110,597 d-020) having about 0.02 inch (about 500 micron) openings and a flat rotor (Fluid Air Part No. 171,144A). The Granumill Jr. was operated at about 10,000 rpm. The flow rate through the mill was not controlled, but the entire volume was poured through the mill in about 30 seconds. Thus, it is estimated that the flow rate was about 10 liters/min.

The resulting milled, frozen solid particles contained in the liquid nitrogen were collected in a bucket. A portion of the liquid nitrogen was allowed to boil off before the microparticles were poured over frozen ethanol, allowed to stand in the ethanol as the ethanol melted, filtered from the ethanol, and lyophilized as described in Example 1A to produce milled microparticles.

The particle size distributions of the milled and unmilled control microparticles were then determined as described in Example 1B. About 19.6 weight percent of the unmilled control microparticles were larger than 106 microns, while an average of about 8.1 weight percent of the homogenized microparticles were larger than 106 microns. Of 21 trials using the Granumill Jr. mill, 18 trials showed an improvement in the percentage of microparticles that were larger than 106 microns. After measuring the particle size distribution, the milled and control microparticles were sieved at 106 microns and the sieve yields (by weight) were determined. Of the 21 trials using the Granumill Jr. mill, 17 out of 20 had higher sieve yields (an average of about 8 weight percent higher, with a range of about 40% higher to 0% or worse) than their respective control (one sample was not sieved due to insufficient material quantities).

### Example 2A

This example describes the production of microparticles containing human growth hormone (hGH). r-hGH was originally obtained from Genentech, Inc. (South San Francisco, Calif.) and subsequently recovered from microparticles produced using a process similar to that described herein. The recovered r-hGH was complexed at a 10:1 molar ratio with zinc by combining the r-hGH with an approximately 54 milliMolar (mM) zice acetate solution to form a mixture with about 20 milligrams r-hGH-zinc complex per millilter of solution. The resulting solution was then spray frozen by spraying the suspension at 400 mL/min through a 2-fluid atomizer (Spray Systems Co. nozzle is air cap part no. 70, fluid cap part no. 2850, Wheaton, Ill.) with 62 slpm of nitrogen gas flow, and co-spraying liquid nitrogen at 30 psi (from 4 Spray Systems Co. nozzles part no. 3004, Wheaton, Ill.) into a tank of liquid nitrogen. The resulting frozen hGH particles were recovered and lypophilized to produce hGH powder.

123 grams of a poly(d,l-lactide-co-glycolide)polymer having 50 mol % d,l-lactide, 50 mol % glycolide, and an acid end group (MEDISORB® 5050 DL PLG 2A polymer; Alkermes, Inc., Cincinnati, Ohio) was dissolved using 615 mL of methylene chloride. The resulting polymer/methylene chloride mixture was then mixed with 22 grams of the hGH powder and 1.5 grams of zinc carbonate. The resulting mixture was homogenized using an Avestin, Inc. EmulsiFlex-C5 (Ontario, Canada). The mixture was passed through the homogenizer 4 times at pressures of about 500 pounds per square inch (psi), about 1000 psi, about 8000 psi, and about 10,000 psi, respectively.

The mixture of hGH powder, zinc carbonate, polymer, and methylene chloride was then spray frozen to produce frozen, solid particles as described in Example 1A. The frozen, solid particles were collected and split into four fractions. Each of the four fractions was then split into two halves. One half of each faction had a portion of the liquid nitrogen removed, was poured over frozen ethanol, was then allowed to stand in the ethanol as the ethanol melted, had the microparticles filtered from the ethanol, and had the microparticles lyophilized as described in Example 1A to produce unmilled control microparticles. The other half was cryogenically milled using a Granumill Jr. as described in Example 1C and then also had a portion of the liquid nitrogen removed, was poured over frozen ethanol, was then allowed to stand in the ethanol as the ethanol melted, had the microparticles filtered from the ethanol, and had the microparticles lyophilized to produce milled microparticles. The particle size distributions of the milled and unmilled microparticles were then determined as described in Example 1B. Thus, the above procedure produced four control fractions and four milled fractions.

### Example 2B

Three fractions of milled microparticles and their respective unmilled control fractions were produced as described in Example 2A (Trials 1, 2, and 3). Each fraction was sieved to exclude microparticles greater than 106 microns. In vivo studies were preformed to evaluate the pharmacokinetic profile of hGH in rats following administration of a single subcutaneous dose of the sieved microparticles.

Male Sprague-Dawley rats (409.8±13.5 grams) were obtained from Charles River Laboratories, Inc. (Wilmington, Mass.). Animals were divided into six test groups. Groups 1-3 received the milled microparticles prepared during Trials 1-3 and Groups 4-6 received the respective control microparticles prepared during Trials 1-3. Each group contained 3 rats.

Each animal was injected subcutaneously once with nominal 50 milligrams of the microparticles. Specifically, the animals were injected subcutaneously into the interscapular region. The injection vehicle was 3% carboxymethylcellulose (‘CMC’) (low viscosity) and 0.1% TWEEN® 20 (i.e., polyoxyethylene 20 sorbitan monooleate, TWEEN® is a trademark of ICI Americas, Inc.) in 0.9% aqueous sodium chloride. Each animal received a dose comprising approximately 50 milligrams of microparticles containing about 6 milligrams of hGH (12% drug load) in a vehicle volume of 0.75 milliliters.

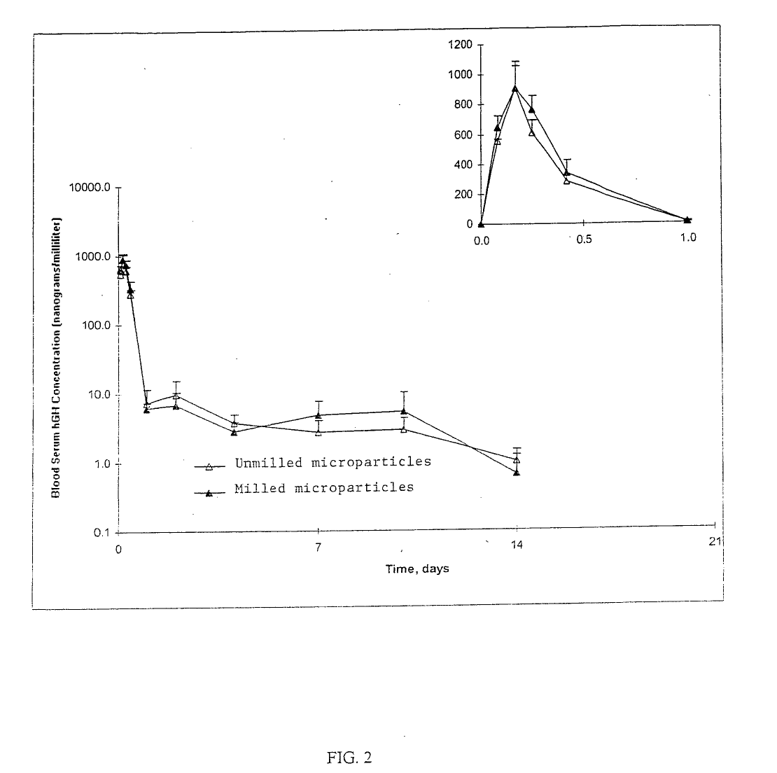

Blood samples were collected via a lateral tail vein after anesthesia with halothane. Blood samples were collected at 2, 4, 6, and 10 hours and then at 1, 2, 4, 7, 10, 14, and 17 days after injection. FIG. 2 shows rat human growth hormone pharmacokinetic (PK) profiles of milled versus unmilled microparticles. The inset of FIG. 2 shows the same data on a time scale of 1 day. The profiles of the Trial 1, 2, and 3 milled microparticles were not significantly different from their respective controls, indicating that cryogenic milling did not affect the in vivo release kinetics of the hGH-loaded microparticles. In addition, there was no significant difference between the profiles of the three control microparticle samples, indicating that Trials 1, 2, and 3 gave reproducible release results.

Table 1 shows C_{MAX} (i.e., maximum hGH concentration in blood serum) and AUC_{0-1 day} (i.e., the area under the curve up to 1 day) for the data shown in FIG. 1.

TABLE 1
Rat hGH pharmacokinetic data for milled and control microparticles

| | Trial 1 | | Trial 2 | | Trial 3 | |
|---|---|---|---|---|---|---|
| | Control | Milled | Control | Milled | Control | Milled |
| C_{MAX} | 818 ± 115.8 | 795 ± 124.4 | 927 ± 71.3 | 705 ± 93 | 684 ± 125 | 707 ± 68.3 |
| (ng/mL) | | | | | | |
| AUC_{0-1 day} | 385 ± 21.4 | 374 ± 19.8 | 371 ± 19.3 | 290 ± 17.1 | 283 ± 55.6 | 301 ± 31.7 |
| (ng*day/mL) | | | | | | |

Table 2 shows analytical test results for milled and control microparticles including hGH for Trials 1, 2, and 3. Particle size was measured using a Coulter LS Particle Size Analyzer (Model 130, Beckman Coulter, Inc. Fullerton, Calif.). Residual methylene chloride was measured by gas chromatography. hGH loading was determined by elemental nitrogen analysis using a CE-440 Elemental Analyzer (Catalog No. 010-00003; Exeter Analytical, Inc., North Chelmsford, Mass.).

Size Exclusion Chromatography (SEC) was used to assess protein degradation. SEC was performed using an isocratic high performance liquid chromatography (HPLC) system with phosphate buffer at 1.0 mL/min using a SUPERDEXM™ 75 HR 10/30 column (Amersham Bioscience, Piscataway, N.J.) containing 13 micron silicon beads. Protein oxidation was determined using HPLC. Protein deamidation was determined using ion exchange chromatography.

Table 2 also shows the results of a 24 hour in vitro release study. 20 mg of microparticles were mixed with 3 mL of HEPES buffer (50 mM HEPES, 95 mM KCl, pH 7.2) at 37° C. The cumulative r-hGH release from the microparticles over time was determined using HPLC or UV-Visible Absorption Spectroscopy. 2 hour and 24 hour accelerated release were determined by mixing 10 mg of microparticles with 1.5 mL of a release buffer for accelerated release at 37° C. The cumulative r-hGH accelerated release from the microparticles over time was determined using HPLC.

TABLE 2
Analytical test results for milled and control microparticles including hGH

| | Trial 1 | | Trial 2 | | Trial 3 | |
|---|---|---|---|---|---|---|
| | Control | Milled | Control | Milled | Control | Milled |
| Mean size (microns) | 26.7 | 29.7 | 24.6 | 20.3 | 22.6 | 22.4 |
| Particles > 120 microns (wt %) | 0% | 0% | 0% | 0% | 0% | 0% |
| Residual methylene chloride (ppm) | ND | ND | ≦30 | ≦30 | ≦30 | ≦30 |
| Residual ethanol | ND | ND | ≦0.3% | ≦0.3% | ≦0.3% | ≦0.3% |
| Zinc load (wt %) | ND | ND | 0.97 | 0.99 | 1.00 | 1.00 |
| Residual water (wt. %) | ND | ND | 3 | 2 | 2 | 2 |
| hGH loading (wt. %) | ND* | ND* | 12.03 | 11.99 | 11.91 | 11.89 |
| Non-aggregated Protein (wt %) | ND | ND | 97 | 97 | 97 | 96 |
| Non-oxidized Protein (wt %) | ND | ND | 98 | 98 | 98 | 98 |
| Non-deamidized Protein (wt %) | ND | ND | 98 | 98 | 98 | 98 |
| 24 hr. in vitro release (%) | 23 | 26 | 35 | 33 | 31 | 31 |
| 2 hr. accelerated release (%) | 59 | 75 | 68 | 64 | 69 | 67 |
| 24 hr. accelerated release (%) | 75** | 88** | 83 | 79 | 84 | 83 |

ND = Not Determined
*Loading assumed to be 12% for testing purposes
**Tested at 23 hours instead of at 24 hours

The collected data shows that the microparticle performance properties, e.g., the in vitro release, are not significantly different between the milled microparticles and the unmilled control microparticles.

### Example 2C

One fraction of milled microparticles and an unmilled control fraction (Trial 4) was produced as described in Example 2A, except that the mixture of hGH, polymer and solvent was spray frozen under conditions favoring a larger particle size, e.g., volume median particle sizes greater than about 50 microns and more than 20 weight percent greater than 106 microns in size. The mixture of hGH, polymer and solvent was spray frozen through an ultrasonic nozzle (Model No. VC130, Sonics & Materials, Inc., Newtown, Conn.) using a syringe pump at a flow rate of 3 mL/min. The microparticles, resulting after the extraction step, had a volume median particle size of about 67 microns. Each fraction was then sieved to exclude microparticles greater than 106 microns. 22 weight percent of the microparticles had a particle size greater than 106 microns.

An in vivo study was performed, as described in Example 2B, to evaluate the pharmacokinetic profile of hGH in rats following administration of a single subcutaneous dose of the sieved Trial 4 microparticles.

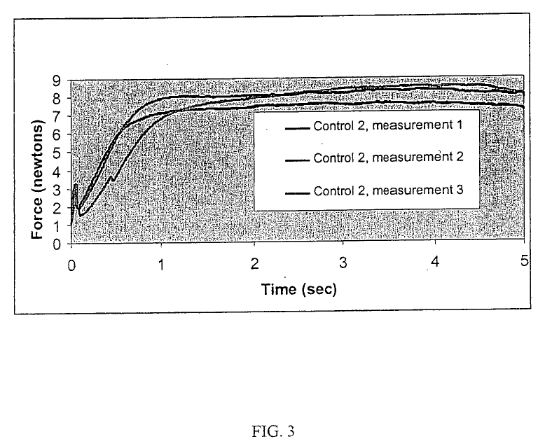

FIG. 3 shows rat human growth hormone pharmacokinetic profiles of milled versus unmilled microparticles. The inset of FIG. 3 shows the same data on a time scale of 1 day. The PK profile of the Trial 4 milled microparticles was not significantly different from the control sample, indicating that cryogenic milling did not significantly affect the in vivo release kinetics of the hGH-loaded microparticles.

Table 3 shows C_{MAX} (i.e., maximum hGH concentration in blood serum) and AUC_{0-1 day} (i.e., the area under the curve up to 1 day) for the data shown in FIG. 2.

TABLE 3
Rat hGH pharmacokinetic data for
milled and control microparticles

Trial 4

| | Control | Milled |
|---|---|---|
| C_{MAX} (ng/mL) | 906 ± 148 | 912 ± 177 |
| AUC_{0-1 day} (ng*day/mL) | 302 ± 32.3 | 349 ± 44.1 |

Because sieve yields are generally about 15% or less, it was postulated that changes in the release profile of the milled microparticles could have been diluted by the majority of microparticles that could have been unaffected by the mill. However, the experiment described in this example demonstrated that cryogenic milling of the larger frozen microparticles did not significantly affect the in vivo release kinetics of the hGH-loaded microparticles.

### Example 3

This example describes the production of placebo microparticles using a microparticle production apparatus similar to that shown in FIG. 1.

500 grams of a poly(d,l-lactide-co-glycolide)polymer having 50 mol % d,l-lactide, 50 mol % glycolide, and an acid end group (MEDISORB® 5050 DL PLG 2A polymer; Alkermes, Inc., Cincinnati, Ohio) was dissolved using 2500 mL of methylene chloride. This mixture was then spray frozen and the solvent was extracted to produce control placebo microparticles using a process similar to that shown in FIG. 1 but that did not contain fragmentation means, e.g., mill 18. Using this apparatus, the mixture was spray frozen to produce frozen solid particles. The mixture was atomized at about 120 mL/minute through a 2-fluid nozzle with a 35 psi nitrogen gas stream (about 160 standard liters per minute) into a liquid nitrogen stream (from 4 nozzles at 30 psi). The nozzles used were as follows: 2-fluid nozzle: fluid cap 2050, air cap 70 m (modified for microparticle production by drilling 8 holes through the air cap to provide for flow of nitrogen gas through the air cap) (Spraying Systems Co., Wheaton, Ill.); and liquid nitrogen nozzles: Model No. 110015 (Spraying Systems Co., Wheaton, Ill.). The spray chamber was in fluid communication with an extraction vessel containing ethanol at a temperature of about −112° C. to about −104° C. The solid particles were transferred to the extraction vessel and retained there for about 2-3 hours as the temperature of the ethanol/solid particle mixture was increased to about −40° C. The liquid nitrogen was removed as it evaporated.

Another mixture of polymer and solvent prepared as described above was then spray frozen using an apparatus similar to that shown in FIG. 1, to produce milled placebo microparticles. The solid particle production section and the extraction section of the apparatus were configured as described above. The mill used was a Granumill Jr. equipped with a screen (Fluid Air Part No. 110,597 d-020) having about 0.02 inch (about 500 micron) openings and a flat rotor (Fluid Air Part No. 171,144A). The Granumill Jr. was operated at about 10,000 rpm. The flow rate of liquid nitrogen into the apparatus was about 1 liter/min and the flow rate of the mixture of polymer and solvent into the apparatus was about 120 mL/min.

A total of three samples of control placebo microparticles and four samples of milled placebo microparticles were produced. The samples of control and milled placebo microparticles were dried using a filter dryer custom manufactured by ITT Sherotec (Simi Valley, Calif.). Drying started under vacuum with the filter dryer jacket set at −25° C. Once the vacuum level in the filter dryer fell below 1750 milliTorr (mTorr), the jacket temperature was stepped up 2° C. every 20 minutes as long as the vacuum remained below 1750 mTorr. Once the temperature of the jacket reached 20° C. (after approximately 2 days of increasing the temperature), the jacket temperature was maintained at 20° C. and the microparticles were held in the dryer until the vacuum fell to below 300 mTorr (approximately 1 day).

Microparticles harvested from the above processes then were sieved to exclude microparticles greater than 106 microns using an 8 inch, 106 micron sieve (VWR International, West Chester, Pa.) used in a sieve shaker (Model SS-15, Gilson Co., Lewis Center, Ohio). Table 4 shows sieve and overall yield data for the control and milled placebo microparticles. “Sprayed Composition” is the mass of polymer in the composition. “Sieved Particles” is the mass of particles passing through the sieve. “Sieve Yield” compares the weight of sieved particles with the weight of the harvested particles. The “Overall Yield” compares the weight of sprayed composition with the weight of the sieved particles and takes into account any losses from hold-up in the production apparatus (e.g., hold-up in the mill section) or other losses (e.g., leaks in the apparatus).

TABLE 4
Sieve and Overall Yield Data

| | Control 1 | Control 2 | Control 3 | Milled 1 | Milled 2 | Milled 3 | Milled 4 |
|---|---|---|---|---|---|---|---|
| Sprayed Composition (g) | 500 | 500 | 500 | 500 | 500 | 500 | 500 |
| Harvested Particles (g) | 463.9 | 466.2 | 463.9 | 298.3 | 436.1 | 427.0 | 418.7 |
| Sieved Particles (g) | 426.1 | 373.2 | 426.1 | 283.5 | 427.6 | 387.6 | 408.3 |
| Retained Particles (g) | 25.3 | 50.9 | 25.3 | 5.75 | 3.8 | 19.6 | 4.9 |
| Sieve yield (wt %) | 91.9 | 80.1 | 91.9 | 95.0 | 98.1 | 90.8 | 97.5 |
| Overall yield (wt %) | 85.2 | 74.6 | 85.2 | 56.7 | 85.5 | 77.5 | 81.7 |

The average sieve and overall yields for the four milled samples were 95.4% and 75.4%, respectively. For the three control samples, the average sieve and overall yields were 87.6% and 81.4%, respectively. Although the milled microparticles exhibited an increase in sieve yield over the control microparticles, there was no improvement in overall yield. The decrease in overall yield was attributed to leaks from the mill and hold-up of fragmented solid particles in the milling apparatus.

### Example 4

It was hypothesized that an insufficient quantity of liquid nitrogen caused the hold-up of fragmented solid particles in the milling apparatus in the experiments described in Example 3. Therefore, this experiment describes experiments wherein microparticles were produced using an additional quantity of liquid nitrogen supplied to the frozen solid particles prior to their introduction to the fragmentation means.

Microparticles were produced using the methods described in Example 3 except that additional liquid nitrogen was introduced to the spray chamber prior to entry of the frozen solid particles to the fragmentation means. The additional nitrogen was added through an additional port such as optional port 16 illustrated in FIG. 1 and described supra. The additional liquid nitrogen was introduced to the spray chamber using an extra nozzle produced by Spraying Systems Co. (Wheaton, Ill.). Table 5 shows the model number of the extra liquid nitrogen nozzle that was used for each trial. Please note that Trial A refers to the Milled 2 trial of Example 3.

TABLE 5
Mill Hold-up in Microparticle Production
Using Additional Liquid Nitrogen

| | Batch | N_{2(liq)} | | Nozzle | Hold- | Hold- | |
|---|---|---|---|---|---|---|---|
| | Size | Nozzle | N_{2(liq)} | Flow* | Up | Up | |
| Trial | (grams) | No. | (PSID) | (gpm) | (mL) | (%) | |
| A | 500 | None | — | — | 125** | about | 5 |
| B | 100 | 3002.5 | 30 | 0.15 | 65 | about | 13 |
| C | 100 | 3007 | 30 | 0.61 | NM | <1 | |
| D | 500 | 3007 | 15 | 0.43 | NM | <1 | |
| E | 500 | 3004 | 30 | 0.35 | 18 | about | 1 |
| F | 500 | 3007 | 30 | 0.61 | NM | <1 | |
| G | 500 | 3007 | 15 | 0.43 | 170 | about | 7 |

| | | | | | | |
|---|---|---|---|---|---|---|
| H | 633 | 3014 | 15 | 0.86 | NM | <1 |

*Nozzle flow is water flow at the pressure used, as provided by the nozzle manufacturer
**Hold-up volume was not measured, but was back calculated from harvest yield loss (25 g) in this lot compared to a control microparticle lot
NM = Not Measurable

As shown in Table 5, an extra liquid nitrogen nozzle that allows a water flow rate of at least about 0.5 gallons per minute (gpm) can be used to reduce or eliminate hold-up of fragmented frozen particles in the mill.

Alternatively or additionally, additional liquid nitrogen can be introduced by increasing flow rates of liquid nitrogen introduced to the spray chamber. For example, the flow rate of liquid nitrogen can be increased through a port such as port 14 illustrated in FIG. 1 and described supra. This approach has the advantage of not requiring the addition of extra liquid nitrogen nozzles in the spray chamber and/or the mill.

To validate this concept, two batches of fragmented microparticles were produced using an apparatus similar to that shown in FIG. 1 but having only the solid particle production section and the fragmentation section. The procedure used was otherwise similar to that described in Example 3. The nozzles used were as follows: 2-fluid nozzle: fluid cap 2050, air cap 70 m (modified for microparticle production by drilling 8 holes through the air cap to provide for flow of nitrogen gas through the air cap) (Spraying Systems Co., Wheaton, Ill.); and liquid nitrogen nozzles: Model Nos. 11004 and 11005 (Spraying Systems Co., Wheaton, Ill.). The 11004 liquid nitrogen nozzle provided an additional 0.64 gpm flow and the 11005 liquid nitrogen nozzle provided an additional 0.84 gpm flow over the previously used 110015 liquid nitrogen nozzle. The first batch, using the 11004 liquid nitrogen nozzle, had a hold-up of about 1.6 grams of fragmented solid particles, less than 1 weight percent. The second batch, using the 11005 liquid nitrogen nozzle, did not have a measurable amount of hold-up. An attempt to produce microparticles as described in Example 3 using the 11004 and 11005 nozzles failed as the heater for the extraction tank jacket was overwhelmed by the ethanol cooling rate and freezing ethanol required the experiment to be halted.

Another trial was performed as described above, but liquid nitrogen nozzles having an additional 0.36 gpm of flow were used (Model No. 11003, Spraying Systems Co., Wheaton, Ill.). In this trial, a liquid nitrogen flow rate of 180 standard liters per minute and a mixture flow rate of 1.50 mL/min were used. The mill in this trial experienced minimal hold-up, as 457.7 grams of microparticles were harvested. The harvested microparticles were then sieved using a 106 micron sieve, as above. The sieve yield was 86.5 weight percent and the total yield was 79.2 weight percent.

### Example 5

This example describes the production of placebo microparticles.

2 kilograms of a poly(d,l-lactide-co-glycolide)polymer having 50 mol % d,l-lactide, 50 mol % glycolide, and an acid end group (MEDISORB® 5050 DL PLG 4A polymer; Alkermes, Inc., Cincinnati, Ohio) was dissolved using 4 liters of acetonitrile. The resulting mixture was frozen into large globules and strands by slowly streaming it into liquid nitrogen. The large globules and strands were kept suspended in about 20 liters of liquid nitrogen and milled using a Granumill Jr. (Fluid Air, Inc.; Aurora, Ill.) equipped with a screen (Fluid Air Part No. 110,597 d-020) having about 0.02 inch (about 500 micron) openings and a flat rotor (Fluid Air Part No. 171,144A). The Granumill Jr. was operated at about 10,000 rpm. The suspension was fed to the mill at about 5 liters/minute. A microparticle slurry was collected from the mill.

Using a freeze/filter dryer similar to that described in U.S. patent application Ser. No. 10/304,058, filed on Nov. 26, 2002, entitled “Method and Apparatus for Filtering and Drying a Product,” incorporated in its entirety herein by reference, the liquid nitrogen was filtered from the microparticles and the microparticles were freeze dried under a vacuum of less than 300 milliTorr for 4 days while the dryer jacket temperature was slowly risen from −50° C. to 25° C. 1528 grams of powder was harvested from the filter dryer. The powder was coarse sieved through a 1/8 inch screen to remove clumps that had formed from melting near the jacket to produce a 1407 gram yield. Scanning electron microscopy indicated that the microparticles were mostly non-spherical, had dense surfaces, and were mostly 100 to 200 microns in diameter.

### Example 6

This example describes experiments measuring the injectability of microparticles prepared in accordance with the present invention.

Samples of milled and unmilled (control) placebo microparticles, were taken from microparticles produced as described in Example 3 as Controls 1, 2, and 3 and Milled 1 and 2. In addition, a sample of unmilled placebo microparticles was produced using a commercial-scale facility for producing microparticles that included atomizing a mixture of 500 grams of a poly(d,l-lactide-co-glycolide)polymer having 50 mol % d,l-lactide, 50 mol % glycolide, and an acid end group (MEDISORB® 5050 DL PLG 2A polymer) and 2500 mL of methylene chloride using a two fluid nozzle; and extracting the methylene chloride in an extraction vessel similar to that shown in FIG. 1 containing ethanol at a temperature of about −112° C. to about −104° C. by retaining the particles there for about 2-3 hours as the temperature of the ethanol/solid particle mixture was increased to about −40° C.

An additional sample of control placebo microparticles was produced using an apparatus similar to that shown in FIG. 1 but that did not contain fragmentation means, e.g., mill 18. The apparatus and procedure used is described in Example 3, supra. However, the mixture that was spray frozen was composed of about 1.6 kilograms of polymer and about 8 liters of solvent.

Each sample of microparticles were sieved after their manufacture to exclude microparticles greater than 106 microns. The microparticles were then suspended in a diluent of 3% carboxymethylcellulose (low viscosity) and 0.1% TWEEN® 20 in 0.9% aqueous sodium chloride at a concentration of 125 milligrams microparticles per milliliter of suspension. The suspension was then filled into 3 mL syringes with 21 Gauge, 1 inch long, thin wall needles (Model No. 305165, Becton, Dickinson, and Co., Franklin Lakes, N.J.). The contents of the syringes were then ejected at 3.3 millimeters/second (0.2 mL/second) for 5 seconds (1 mL total ejection) and the force required to maintain this ejection force was measured over time using a Texture Analyser (Model no. TA-XT2i, Stable Micro Systems, Ltd., United Kingdom). Clogs or partial clogs were indicated by a spike in this measured force. Ejection force for each lot of microparticles suspended in diluent was measured three times.

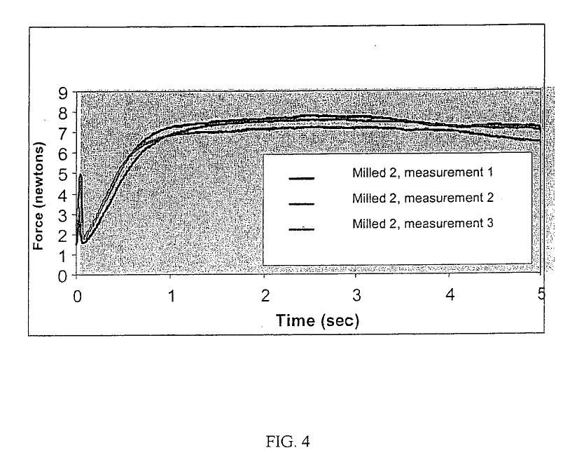

FIGS. 3 and 4 show typical ejection force profiles for a control lot and for a milled lot, respectively. The figures show that there were no ejection force spikes and thus there was no clogging or partial clogging of the syringe needle. This experiment did not show any difference between the force required for ejection of milled microparticles and unmilled control microparticles. In addition, while there was some lot-to-lot variability, there was no significant difference between the mean and maximum force required for ejecting milled microparticles versus ejecting unmilled control microparticles.

While this invention has been particularly shown and described with references to preferred embodiments thereof, it will be understood by those skilled in the art that various changes in form and details may be made therein without departing from the scope of the invention encompassed by the appended claims.

1. A method for forming microparticles, comprising the steps of:
(a) fragmenting solid particles that include a biologically active agent, a biocompatible polymer and a solvent, thereby producing fragmented solid particles; and
(b) separating the solvent from the fragmented solid particles, thereby forming the microparticles.

2. The method of claim 1 further comprising the steps of forming a mixture of the biologically active agent, the biocompatible polymer and the solvent, and freezing the mixture to form the solid particles.

3. The method of claim 2 wherein freezing the mixture to form the solid particles includes atomizing the mixture to form droplets, and freezing the droplets.

4. The method of claim 3 wherein the droplets are microdroplets.

5. The method of claim 3 wherein the mixture is atomized into or near a cryogenic fluid.

6. The method of claim 5 wherein the cryogenic fluid is liquid nitrogen.

7. The method of claim 2 wherein freezing the mixture to form the solid particles includes forming frozen strands of the mixture, thereby forming the solid particles.

8. The method of claim 1 wherein the biocompatible polymer is biodegradable.

9. The method of claim 1 wherein the biocompatible polymer is at least one member selected from the group consisting of poly(lactide)s, poly(glycolide)s, poly(lactide-co-glycolide)s, poly(lactic acid)s, poly(glycolic acid)s, polycarbonates, polyesteramides, polyanhydrides, poly(amino acids), polyorthoesters, polyacetals, polycyanoacrylates, polyetheresters, polycaprolactone, poly(dioxanone)s, poly(alkylene alkylate)s, polyurethanes, and blends and copolymers thereof.

10. The method of claim 9 wherein the biocompatible polymer is a poly(lactide-co-glycolide).

11. The method of claim 1 wherein the biologically active agent is at least one member selected from the group consisting of proteins, immunoglobulin proteins, interleukins, interferons, erythropoietin, antibodies, cytokines, hormones, antigens, growth factors, nucleases, tumor enzymes, tumor suppression genes, antisense molecules, antibiotics, anesthetics, sedatives, cardiovascular agents, antitumor agents, antineoplastics, antihistamines and vitamins.

12. The method of claim 1 wherein the biologically active agent is human growth hormone.

13. The method of claim 1 wherein the solvent is selected from the group consisting of methylene chloride, chloroform, ethyl acetate, methyl acetate, acetone, acetic acid, acetonitrile, dimethylsulfoxide, methyl ethyl ketone and toluene.

14. The method of claim 1 wherein the solvent is present in the solid particles at an average concentration of at least about 30 weight percent.

15. The method of claim 1 wherein the solid particles have a particle size of about 500 microns to about 3 inches prior to fragmenting.

16. The method of claim 1 wherein the solid particles have a particle size of less than or equal to about 200 microns prior to fragmenting.

17. The method of claim 1 wherein the solid particles are fragmented by milling.

18. The method of claim 1 wherein the solid particles are fragmented using an impact mill or a screening mill.

19. The method of claim 18 wherein the solid particles are fragmented by impacting the solid particles with a rotor and passing the impacted solid particles through a screen.

20. The method of claim 1 wherein the solid particles are fragmented while suspended in a cryogenic fluid.

21. The method of claim 20 wherein the cryogenic fluid is liquid nitrogen.

22. The method of claim 1 wherein the solid particles are fragmented while suspended in a polymer non-solvent, and wherein the temperature of the polymer non-solvent is below the melting temperature of the solvent contained in the solid particles.

23. The method of claim 1 wherein the solvent is separated from the fragmented solid particles by drying.

24. The method of claim 23 wherein drying includes sublimation of the solvent from the fragmented solid particles.

25. The method of claim 1 wherein the solvent is separated from the fragmented solid particles by extracting the solvent into a polymer non-solvent.

26. The method of claim 25 wherein the polymer non-solvent is ethanol.

27. The method of claim 25 further comprising the step of vacuum drying the microparticles.

28. A method for producing microparticles, comprising the steps of:
(a) forming a mixture including a biologically active agent, a biocompatible polymer, and a solvent;
(b) atomizing the mixture to form droplets and freezing the droplets, thereby producing solid particles;
(c) fragmenting the solid particles, thereby forming fragmented solid particles; and
(d) separating the solvent from the fragmented solid particles, thereby forming the microparticles.

29. The method of claim 28 wherein the mixture is atomized into a cryogenic fluid.

30. The method of claim 28 wherein the mixture also contains one or more excipients.

31. The method of claim 28 wherein the biocompatible polymer is at least one member selected from the group consisting of poly(lactide)s, poly(glycolide)s, poly(lactide-co-glycolide)s, poly(lactic acid)s, poly(glycolic acid)s, polycarbonates, polyesteramides, polyanhydrides, poly(amino acids), polyorthoesters, polyacetals, polycyanoacrylates, polyetheresters, polycaprolactone, poly(dioxanone)s, poly(alkylene alkylate)s, polyurethanes, and blends and copolymers thereof.

32. The method of claim 28 wherein the biologically active agent is selected from the group consisting of proteins, immunoglobulin proteins, interleukins, interferons, erythropoietin, antibodies, cytokines, hormones, antigens, growth factors, nucleases, tumor enzymes, tumor suppression genes, antisense molecules, antibiotics, anesthetics, sedatives, cardiovascular agents, antitumor agents, antineoplastics, antihistamines and vitamins.

33. The method of claim 28 wherein the solvent is present in the solid particles at an average concentration of at least about 30 weight percent.

34. The method of claim 28 wherein the solid particles have a particle size of less than or equal to about 200 microns prior to fragmenting.

35. The method of claim 28 wherein the solid particles are fragmented using an impact mill or a screening mill.

36. The method of claim 35 wherein the solid particles are fragmented by impacting the solid particles with a rotor and passing the impacted solid particles through a screen.

37. The method of claim 28 wherein the solid particles are fragmented while suspended in a cryogenic fluid.

38. The method of claim 28 wherein the solvent is separated from the fragmented solid particles by extracting the solvent into a polymer non-solvent.

39. A method for producing an injectable pharmaceutical composition comprising the steps of:
(a) forming a mixture including a biologically active agent, a biocompatible polymer, and a solvent;
(b) atomizing the mixture to produce droplets and freezing the droplets, thereby producing solid particles;
(c) fragmenting the solid particles, thereby forming fragmented solid particles;
(d) separating the solvent from the fragmented solid particles, thereby forming microparticles;
(e) size-separating microparticles unsuitable for administration by injection from the microparticles, thereby producing an injectable microparticle population; and
(f) forming a mixture of the injectable microparticle population and a physiologically acceptable diluent, thereby forming the injectable pharmaceutical composition.

40. The method of claim 39 wherein the solvent is present in the solid particles at an average concentration of at least about 30 weight percent.

41. The method of claim 39 wherein the solid particles are fragmented using an impact mill or a screening mill.

42. The method of claim 41 wherein the solid particles are fragmented by impacting the solid particles with a rotor and passing the impacted solid particles through a screen.

43. The method of claim 39 wherein the solid particles are fragmented while suspended in a cryogenic fluid.

44. The method of claim 39 wherein size-separating microparticles unsuitable for administration by injection from the microparticle population includes sieving.

45. An apparatus for producing microparticles, comprising:
(a) a solid particle production section including a fluid atomizer, at least one port for introducing a cryogenic fluid, and a spray chamber;
(b) a fragmentation section including a solid particle fragmentation means; and
(c) an extraction section including an extraction vessel containing a polymer non-solvent;
wherein the solid particle production section is joined in fluid communication with the fragmentation section and the fragmentation section is joined in fluid communication with the extraction section.

46. The apparatus of claim 45 wherein the spray chamber of the solid particle production section has an upper-portion that includes the fluid atomizer and at least one port for introducing a cryogenic fluid and a lower-portion that includes a solid particle outlet and at least one port for introducing a cryogenic fluid.

47. The apparatus of claim 46 wherein the solid particle outlet of the spray chamber is in fluid communication with the fragmentation section.
